(19) United States
(12) Patent Application Publication
(10) Pub. No.: US 2025/0009573 A1
(43) Pub. Date: Jan. 9, 2025

(54) QUICKBANDAGE

(71) Applicant: RAM P PRABHU, North Billerica, MA (US)

(72) Inventor: RAM P PRABHU, North Billerica, MA (US)

(21) Appl. No.: 18/219,681

(22) Filed: Jul. 9, 2023

(51) Int. Cl. A61F 15/00

(52) U.S. Cl. CPC A61F 15/002

(57) ABSTRACT

This invention delivers bandages of equal size attached to a wax paper roll. Each bandage incorporates a Tab to pull it off the wax paper for use to cover and protect small wounds. The Tab also facilitates pulling the bandage off one's skin when needed.

### CROSS REFERENCE OF RELATED APPLICATIONS

Not Applicable

### STATEMENT REGARDING FEDERALLY SPONSORED RESEARCH OR DEVELOPMENT

Not Applicable

### REFERENCE TO A SEQUENCING LISTING A LARGE TABLE OR A COMPUTER LISTING APPENDIX OR READ ONLY OPTICAL DISC

Not Applicable

### BACKGROUND OF THE INVENTION

US Patent Classification Field: Class 5 Pharmaceutical and other preparations for medical or veterinary purposes.

Current State of Bandage Art to Protect Small Scratches and Bruises:

When I recently had my Covid-19 shot there were over 50 people waiting in line to get the shot. After I received a shot on my arm the nurse asked me to hold a piece of cotton over the injection area. She then turned around and teared off a couple of layers of protective materials to get a bandage out of a strip and used it to cover the injection area on my arm.

As I watched the nurse giving shots to next few people in turn I realized that the nurse spent a lot more time in getting a bandage out of its protective wraps of material as compared to the time she spent in actually giving the injection.

When it came time to remove the bandage off my arm I had to first slide my finger nail between one edge of the bandage and my skin to ‘scratch’ out a starting edge of the bandage so I could grab and hold the edge and pull the bandage off.

### BRIEF SUMMARY OF THE INVENTION

A bandage in the market today to cover a small wound or injection area is mostly available as a single strip made up of a single bandage wrapped around with multiple layers of protective materials which need to be carefully teared off first to access the bandage. When such a bandage needs to be removed most times one needs to scratch out a leading edge to hold and pull the bandage off one's skin.

This invention delivers multiple bandages of the same size attached to a wax paper roll. Each bandage has a Tab attached. The Tab allows a bandage to be pulled off quickly from the wax paper roll and use it to cover a small wound or injection area. The Tab also provides a leading edge for easy and safe removal of the bandage from one's skin.

### BRIEF DESCRIPTION OF SEVERAL VIEWS OF DRAWING

FIG. 1: Depicts a sterilized cotton Strip (A) with an adhesive on one side only.

FIG. 2: Depicts a sterilized cotton Pad (B) to cover and protect small wound.

FIG. 3: Depicts a Tab (C) made of wax paper.

FIG. 4: Depicts a single bandage with adhesive side facing up with the cotton Strip (A), the cotton Pad (B) and the wax paper Tab (C) attached.

FIG. 5: Depicts a wax paper roll with perforations and multiple bandages of same size attached.

FIG. 6: Depicts a user pulling a bandage off the wax paper roll using the Tab (C).

FIG. 7: Depicts a patient with a bandage attached over an injection area with half of the Tab (C) hanging out unattached.

FIG. 8: Depicts a patient removing the bandage off her arm using the unattached half of the Tab (C) to pull.

### DETAILED DESCRIPTION OF THE INVENTION

Following are details of how to create and use the proposed bandage. A total of fifty (50) bandages adhered to a wax paper roll to be made available to the end user.

### Creating the Proposed Bandage

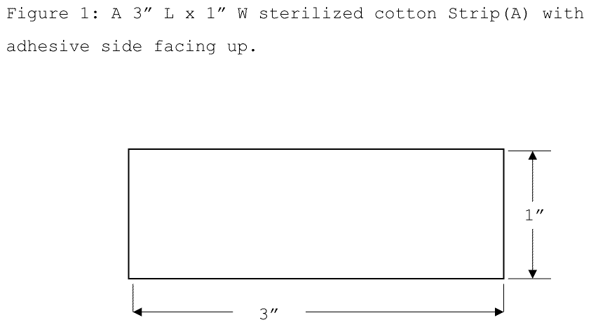

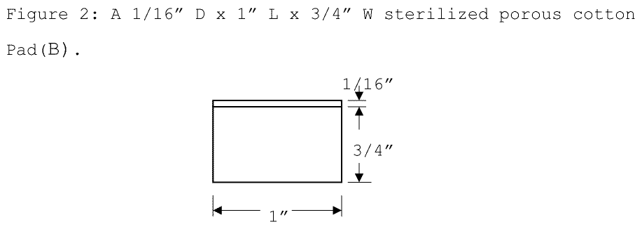

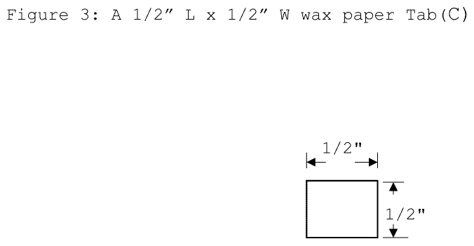

Each bandage is made separately and has three components assembled together:
1. A 3″ L×1″ W sterilized cotton Strip (A) with an adhesive on one side only. Refer to FIG. 1.
2. A 1/16″ D×1″ L×¾″W sterilized cotton Pad (B). Refer to FIG. 2.
3. A ½″×½″ square wax paper Tab (C). Refer to FIG. 3.

First on a clean sterilized work table the cotton Strip (A) is placed longitudinally with adhesive side facing up.

Next the cotton Pad (B) is adhered longitudinally at the center of Strip (A) such that there is a margin of 1″ between left edges of Pad (B) and Strip (A) and a crosswise margin of ⅛″ between the top edge of Strip (A) and top edge of Pad (B).

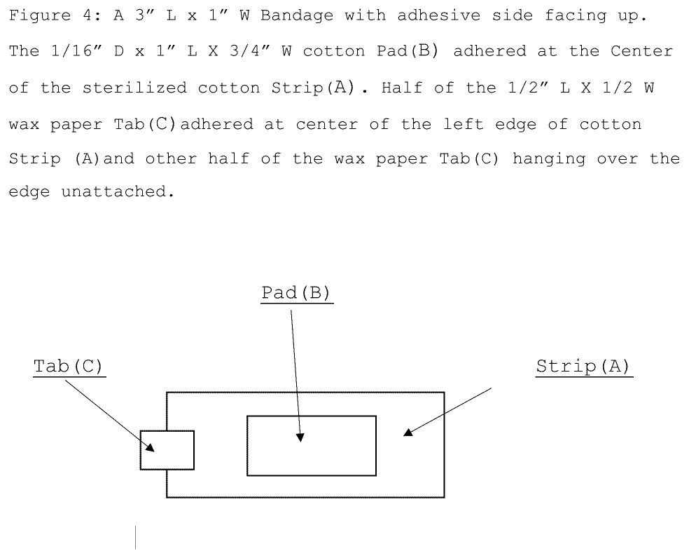

The wax paper Tab (C) is then adhered at the center of the left edge of Strip (A) such that ¼″ of the Tab (C) is attached to Strip (A) and the other ¼″ of Tab (C) is hanging out unattached. Refer to FIG. 4.

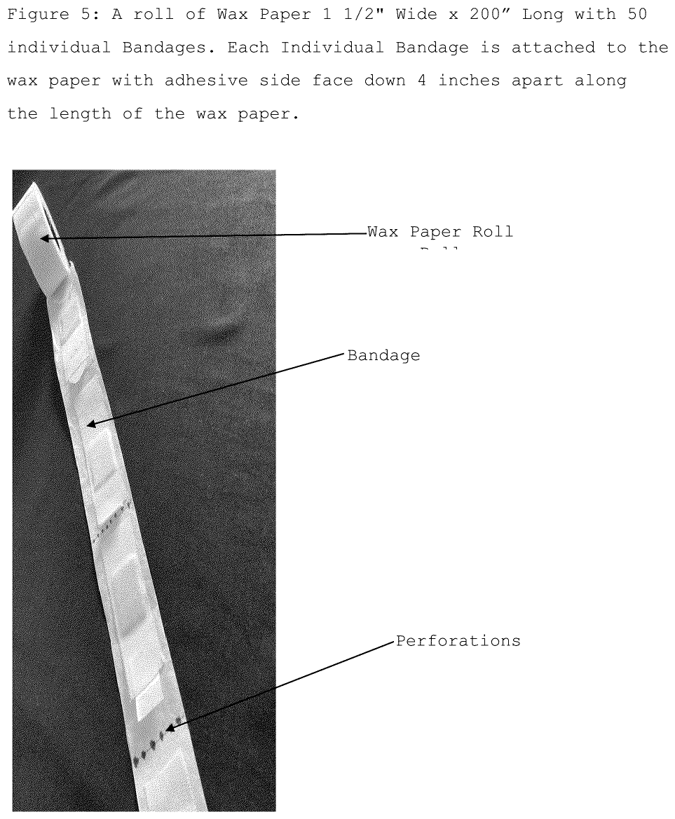

Each bandage is then adhered face down 4 inches apart to a 1½″ W×200″ L wax paper roll. There will be 50 bandages attached over the length of the wax paper roll. The wax paper roll will have perforations 4 inches apart longitudinally. Refer to FIG. 5.

### How to Use the Proposed Bandage

When needed to cover and protect a small bruised area the user first pulls the leading edge of wax paper roll that has a bandage attached to it.

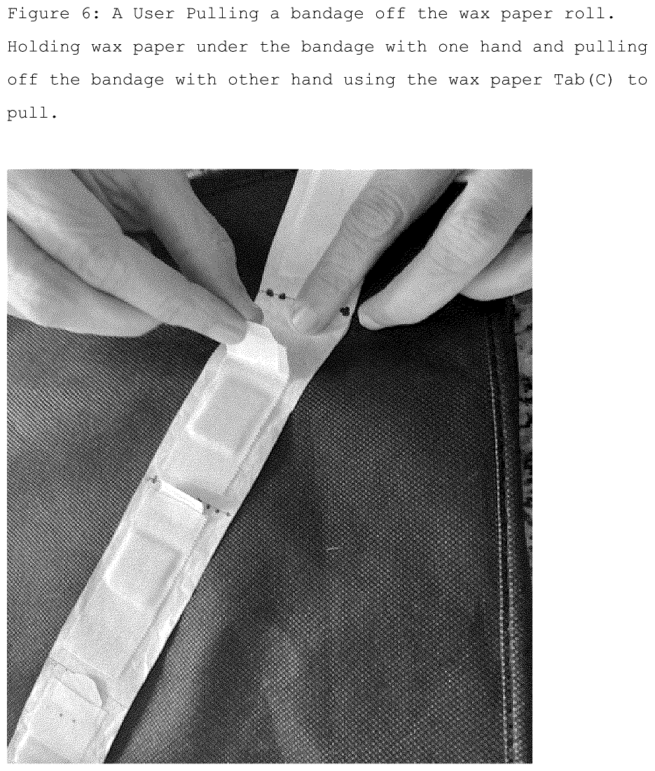

The user then holds down a part of the wax paper underneath the bandage with one hand and then pulls off the bandage with other hand using the wax paper Tab. Refer to FIG. 6.

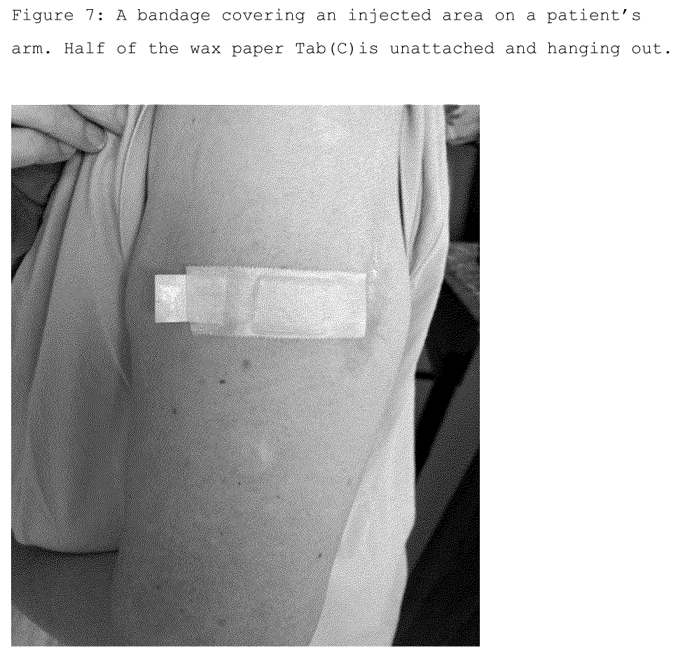

The bandage is then adhered to one's skin such that the Pad (B) completely covers the bruised area. Refer to FIG. 7.

When the bandage needs to be removed from one's skin the user first holds down exposed area of his skin few inches away from the bandage with one hand, holds the wax paper Tab (C) with the other hand and uses it to pull the bandage off.

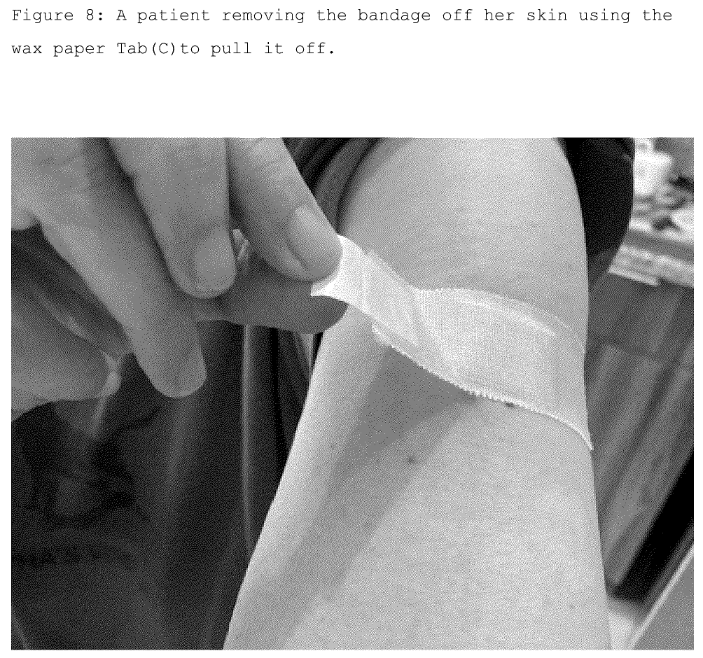

Refer to FIG. 8.

After pulling a bandage off the wax paper roll the piece of wax paper that was attached under the bandage can be teared off at the perforations and discarded.

1. Faster access to sterilized bandages saving valuable time to cover and protect injunction areas and small wounds.

2. Less effort needed to pull off a bandage from one's skin.
